(19) United States
(12) Patent Application Publication
(10) Pub. No.: US 2013/0131497 A1
(43) Pub. Date: May 23, 2013

(54) PLATFORM, ASSEMBLY, AND METHOD OF POSITIONING AN RF COIL

(71) Applicants: ANTON M. LINZ, WAUKESHA, WI (US); PETER S. CRANDALL, WAUKESHA, WI (US); GARY V. MCBROOM, WAUKESHA, WI (US)

(72) Inventors: ANTON M. LINZ, WAUKESHA, WI (US); PETER S. CRANDALL, WAUKESHA, WI (US); GARY V. MCBROOM, WAUKESHA, WI (US)

(73) Assignee: GENERAL ELECTRIC COMPANY, SCHENECTADY, NY (US)

(21) Appl. No.: 13/302,857

(22) Filed: Nov. 22, 2011

(51) Int. Cl. A61B 5/055; A61G 13/12; A61G 13/10

(52) U.S. Cl. 600/415; 5/601

(57) ABSTRACT

A platform configured to be mounted onto a body support. The platform includes a body portion having an underside and an opposite top side. The underside is configured to face the body support, and the top side is configured to support a patient. The body portion also has a coil-receiving recess along the underside. The coil-receiving recess is configured to form a coil-receiving gap when the platform is mounted onto the body support. The coil-receiving gap is sized and shaped to permit a radio-frequency (RF) coil to be positioned therein.

### BACKGROUND OF THE INVENTION

The subject matter disclosed herein relates generally to medical imaging systems and more particularly, to body supports that support a patient during an imaging session.

Patients can be imaged using a wide variety of different imaging technologies. Medical imaging systems may include magnetic resonance imaging (MRI), computer tomography (CT), positron emission tomography (PET), single photon emission computed tomography (SPECT), x-ray imaging, and others. Imaging systems typically include field-of-views (FOVs) where a patient is positioned to be imaged. On some occasions, a patient is imaged using multiple modalities. For example, x-ray imaging can be used to guide invasive devices and MRI can be used to monitor the results of therapy in the surrounding tissues.

In some applications, it may be necessary to move the patient from a first imaging system (e.g., MR imaging system) to a second imaging system (PET/CT imaging system) using a transporter that carries a body support such as a transfer board. The transfer board is configured to slide into the imaging systems while the patient lies on the transfer board. For example, when the transporter is docked with the first imaging system, the transfer board can be moved axially along a cradle of the first imaging system to position the patient within the FOV of the first imaging system. After the imaging session of the first imaging system, the patient may be moved onto the transporter and then moved to the second imaging system. The patient is then positioned within the second imaging system by moving the transfer board onto a cradle of the second imaging system.

MR imaging can include using a localized radio-frequency (RF) coil that is configured for a particular part of the human anatomy. The RF coil is positioned proximate to the patient (e.g., proximate to the torso or neck/head) during the imaging session. By way of example, a RF coil may be placed directly underneath the patient for MR imaging. However, it is generally undesirable for the RF coils to remain beneath the patient during PET/CT imaging because the materials of the RF coils may cause artifacts in the PET/CT images. If possible, the patient is moved or is asked to move so that the RF coil may be removed before PET/CT imaging. However, this patient movement may present challenges to registering the MR images and the PET/CT images.

### BRIEF DESCRIPTION OF THE INVENTION

In one embodiment, a platform configured to be mounted onto a body support is provided. The platform includes a body portion having an underside and an opposite top side. The underside is configured to face the body support, and the top side is configured to support a patient. The body portion also has a coil-receiving recess along the underside. The coil-receiving recess is configured to form a coil-receiving gap when the platform is mounted onto the body support. The coil-receiving gap is sized and shaped to permit a radio-frequency (RF) coil to be positioned therein.

Optionally, the platform has a weight and size that permit the platform to be carried and mounted onto the body support by a single individual. Also optionally, the body portion may have a substantially uniform cross-section along the coil-receiving recess.

In another embodiment, a platform assembly for a body support is provided. The platform assembly includes a platform configured to be mounted onto a support surface of the body support. The platform has an underside that faces the support surface, and an opposite top side that is configured to support a patient. A coil-receiving gap exists between the underside and the support surface when the platform is mounted onto the body support. The platform assembly also includes a radio-frequency (RF) coil that is configured to be positioned between the underside and the support surface in the coil-receiving gap. The RF coil is configured to communicatively couple to a magnetic resonance (MR) imaging system.

In yet another embodiment, a method of positioning a radio-frequency (RF) coil for imaging a patient is provided. The method includes positioning a platform over a body support having a support surface. The platform includes an underside that faces the support surface. A coil-receiving gap exists between the underside and the support surface. The method also includes positioning the RF coil within the coil-receiving gap between the underside and the support surface. The RF coil is communicatively coupled to a magnetic resonance (MR) imaging system.

### BRIEF DESCRIPTION OF THE DRAWINGS

FIG. 1 illustrates an exemplary medical transporter that is used to transfer a body support between different imaging systems.

FIG. 2 is a perspective view of a platform formed in accordance with one embodiment that may be used with the body support of FIG. 1.

FIG. 3 is a perspective view of an underside of the platform of FIG. 2.

FIG. 4 illustrates the platform in relation to radio-frequency (RF) coils that may be used with the platform.

FIG. 5 is a side view of the platform with the patient lying thereon and the RF coils positioned for imaging.

FIG. 6 is a flowchart illustrating a method for imaging a patient in accordance with one embodiment.

FIG. 7 is a flowchart illustrating another method for imaging a patient in accordance with one embodiment.

FIG. 8 is a flowchart illustrating a method for imaging a patient in accordance with one embodiment.

### DETAILED DESCRIPTION OF THE INVENTION

The foregoing summary, as well as the following detailed description of certain embodiments of the present invention, will be better understood when read in conjunction with the appended drawings. As used herein, an element or step recited in the singular and proceeded with the word “a” or “an” should be understood as not excluding plural of said elements or steps, unless such exclusion is explicitly stated. Furthermore, references to “one embodiment” or “an embodiment” of the present invention are not intended to be interpreted as excluding the existence of additional embodiments that also incorporate the recited features. Moreover, unless explicitly stated to the contrary, embodiments “comprising” or “having” an element or a plurality of elements having a particular property may include additional elements not having that property.

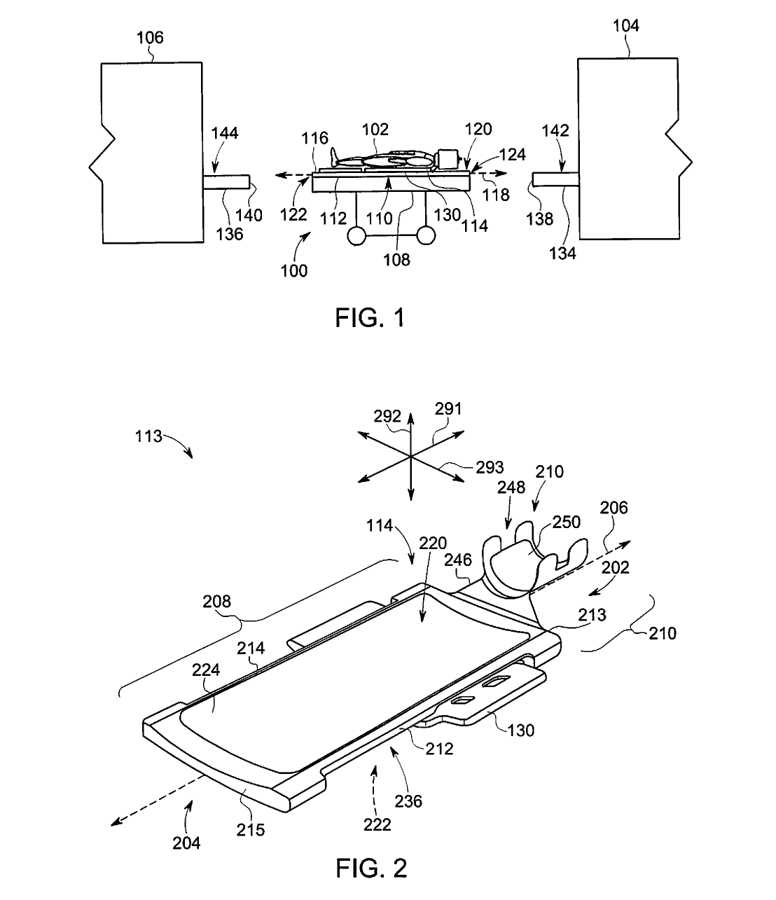

FIG. 1 is a side view of an exemplary medical transporter 100 formed in accordance with one embodiment that is used to transfer a patient 102 between different imaging systems, such as first and second imaging systems 104, 106. Only representative portions of the imaging systems 104, 106 are shown in FIG. 1. The transporter 100 includes a transporter base 108 and a movable support assembly 110. The transporter base 108 is configured to hold the support assembly 110 when the support assembly 110 is transferred between the imaging systems 104, 106. The support assembly 110 can be slidably engaged to the transporter base 108.

In an exemplary embodiment, the support assembly 110 includes a body support, such as a transfer board 112, and a platform 114 that is mounted onto the transfer board 112. In some embodiments, the transfer board 112 is a pre-existing support that is capable of supporting a patient without the platform 114 during an imaging session. As shown, when the platform 114 is mounted to the transfer board 112, the platform 114 is configured to receive the patient 102.

The transfer board 112 includes an elongated transfer body 116 having first and second ends 122, 124, respectively, with a longitudinal axis 118 extending therebetween. The transfer body 116 has a patient or support surface 120 that extends along the longitudinal axis 118. The transfer body 116 is configured to have a patient lie directly over the support surface 120 during imaging sessions in which the platform 114 is not used or indirectly over the support surface 120 during imaging sessions in which the platform is used. Although not shown, the transfer body 116 also includes a bottom surface that interfaces with the transporter base 108. The transfer body 116 can be slidably engaged to the transporter base 108. For example, the transporter base 108 may include guide rails that engage tracks along the transfer body 116. Alternatively, the transporter base 108 may include guide tracks that engage guide rails of the transfer body 116. In other embodiments, rollers or bearings may be used to slidably engage the transfer board 112 to the transporter base 108.

The first and second imaging systems 104, 106 may be any type of imaging system including a multi-modality imaging system. In an exemplary embodiment, the imaging system 104 is a Magnetic Resonance (MR) imaging system and the imaging system 106 is a dual-modality imaging system that is capable of Positron Emission Tomography (PET) imaging and Computed Tomography (CT) imaging in a common gantry. However, the imaging system 106 may be other types of imaging systems, including X-Ray radiography, fluoroscopy, Single Photon Emission Computed Tomography (SPECT) and/or any other type of imaging modality that is capable of generating images of a region of interest (ROI) of a patient. Generally, embodiments described herein may be used for various purposes with multiple imaging systems in which one of the imaging systems is an MR imaging system. In particular embodiments, the imaging systems 104, 106 are for imaging human subjects. However, the imaging systems 104, 106 may also be used for veterinary purposes. As used herein, the term “patient” may refer to a human patient or an animal.

The imaging systems 104, 106 may include respective patient tables or cradles 134, 136 having corresponding docking interfaces 138, 140. The patient cradles 134, 136 have cradle surfaces 142, 144, respectively, that are configured to receive the transfer board 112. In some cases, the patient cradles 134, 136 are capable of imaging the patient without the support assembly 110. The docking interfaces 138, 140 are configured to engage the transporter 100 and permit the support assembly 110 to slide onto the patient cradle 134, 136 or, more particularly, onto the cradle surfaces 142, 144, The cradle 134, 136 may slidably engage the transfer body 116 in a similar manner as described above between the transfer body 116 and the transporter base 108. To this end, the docking interfaces 138, 140 and the transporter 100 are configured to couple to each other and facilitate a smooth transfer of the support assembly 110 to the cradle surface 142, 144.

In the illustrated embodiment, the body support is the transfer board 112, which is then moved onto a patient cradle 134 or 136 (described in greater detail below). However, the body support may be any elongated structure that supports a patient near the FOV during an imaging session. For example, in alternative embodiments, the body support could be the patient cradles 134, 136 (or other imaging table) and the platform 114 could be directly mounted to the patient cradle 134 or 136 without the transfer board 112 therebetween. Like the transfer board 112 and the patient cradles 134, 136, body supports may include an elongated structure having a support surface that is configured to support the platform 114 and/or a patient.

As shown, the platform 114 can be a low-profile accessory that is configured to be mounted and removably secured to the transfer board 112. As will be described in greater detail below, the platform 114 permits a removable RF coil 130 to be positioned below the patient 102 without requiring the patient 102 to move in order to position the RF coil 130. The platform 114 is removably secured to the transfer board 112. As used herein, the term “removable” when used to modify “position,” “couple,” “engage,” “mount,” or “secure” means the components may be readily separated without destroying or significantly damaging either component. Two components are readily separable when the components can be separated without significant effort and within a reasonable period of time for its intended use. For example, it may be necessary for an operator of the imaging systems 104, 106 to mount and demount the platform 114 to the transfer board 112 multiple times (e.g., at least two) within a day or shift. As such, the transfer board 112 may be used to image an ROI before the platform 114 is secured to the transfer board 112, while the platform 114 is secured to the transfer board 112, or after the platform 114 is removed from the transfer board 112.

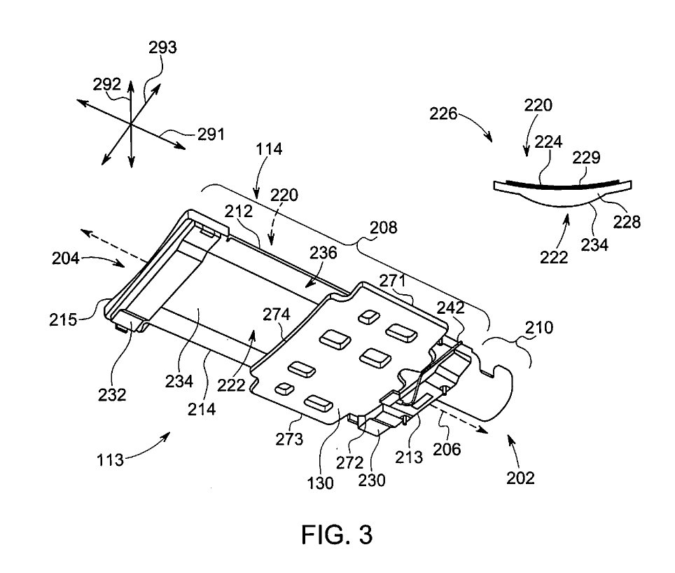

FIG. 2 is a top perspective view of a platform assembly 113, and FIG. 3 is a bottom perspective view of the platform assembly 113. The platform assembly 113 includes the platform 114 and the RF coil 130. In some embodiments, the platform assembly 113 may include the transfer board 112. As shown, the platform 114 is oriented with respect to reference axes 291-293 including a longitudinal axis 291, a vertical axis 292, and a horizontal axis 293. The platform 114 includes a leading end 202, a trailing end 204, and a platform axis 206 extending therebetween. The platform axis 206 extends parallel to the longitudinal axis 291. In some cases, the leading end 202 is configured to be inserted into the imaging bore (not shown) of an imaging system prior to the trailing end 204. The head of the patient 102 (FIG. 1) is configured to lay proximate to the leading end 202.

The platform 114 includes a body portion 208 that extends between the leading and trailing ends 202, 204. The platform 114 may also include a head extension 210 that is coupled to the body portion 208 proximate to the leading end 202. However, the head extension 210 is optional and the platform 114 may comprise only the body portion 208. The body portion 208 may have a rectangular or panel-like shape that includes a plurality of sides 212-215 including longitudinal sides 212, 214 that extend along the platform axis 206 between the leading and trailing ends 202, 204 and also body sides 213, 215 that are proximate to the leading and trailing ends 202, 204, respectively.

The body portion 208 has a top or body side 220 that is configured to receive the patient 102 and an underside 222 that is configured to interface with the support surface 120 of the transfer board 112. The top side 220 and the underside 222 face in substantially opposite directions along the vertical axis 292. In an exemplary embodiment, the platform 114 is configured to be used in MR imaging protocols as well as PET and CT imaging protocols. As such, the platform 114 may include non-conductive materials having a substantially low and uniform density. The materials may be substantially radiation transparent. For example, the platform 114 may include a composite body material 228 (cross-section 226 shown in FIG. 3) that provides structural integrity for the platform 114 and, optionally, a padding material 229 (FIG. 3) (e.g., foam) that is shaped as a pad 224 along the top side 220.

As shown in the cross-section 226 that is taken along the platform axis 206 through a mid-section of the body portion 208, the platform 114 may include only the body material 228 and the padding material 229 of the pad 224. The cross-section 226 is taken perpendicular to the platform axis 206 and may be representative of the body portion 208 along the coil-receiving recess 236. The body portion 208 can have a substantially uniform cross-section along the coil-receiving recess 236. In particular embodiments, the platform 114 is designed to be MR compatible while maintaining low X-ray and gamma ray attenuation for PET/CT imaging.

As shown in FIG. 3, the platform 114 includes first and second bases 230, 232 that are located proximate to the leading and trailing ends 202, 204, respectively. The first and second bases 230, 232 project away from the underside 222 in a direction along the vertical axis 292. In the illustrated embodiment, the first and second bases 230, 232 extend substantially entirely between the longitudinal sides 212, 214 along the horizontal axis 293. However, the first and second bases 230, 232 may extend only partially between the longitudinal sides 212, 214. In other embodiments, the first and second bases 230, 232 may take other forms and shapes. For example, each of the first and second bases 230, 232 may include two separate members or parts that are located proximate to the longitudinal sides 212, 214. In other words, the platform 114 could include four separate legs that are configured to engage the transfer board 112.

In an exemplary embodiment, the first and second bases 230, 232 are separate parts that are coupled to the body portion 208. The first and second bases 230, 232 may constitute a different type of material. For example, the first and second bases 230, 232 may be formed from another material that is non-conductive and has a substantially low and uniform density. The first and second bases 230, 232 can be shaped to complement the support surface 120. In an exemplary embodiment, the first and second bases 230, 232 are secured to the body portion 208 using fasteners. In alternative embodiments, the first and second bases 230, 232 may be secured using an adhesive or even without using an attachment mechanism, such as through an interference fit (or frictional engagement) with the body portion 208.

As shown in FIG. 3, the underside 222 includes a longitudinal contoured section 234 that extends between the leading and trailing ends 202, 204. The contoured section 234 extends toward the transfer board 112 (FIG. 1) when the platform 114 is removably secured thereto. The contoured section 234 constitutes a portion of the platform 114 that has an increased thickness relative to other portions. The increased thickness may be configured to provide additional structural integrity to the platform 114.

The platform 114 includes a coil-receiving recess 236 along the underside 222 between the first and second bases 230, 232. The coil-receiving recess 236 is defined between the bases 230, 232 and beneath the underside 222. As will be described in greater detail below, the coil-receiving recess 236 becomes a coil-receiving gap or gap 240 (FIG. 5) when the platform 114 is mounted to the transfer board 112. The coil-receiving recess 236 (and the coil-receiving gap 240) is sized and shaped to receive the RF coil 130. In the illustrated embodiment, the coil-receiving recess 236 is accessible from the longitudinal sides 212, 214.

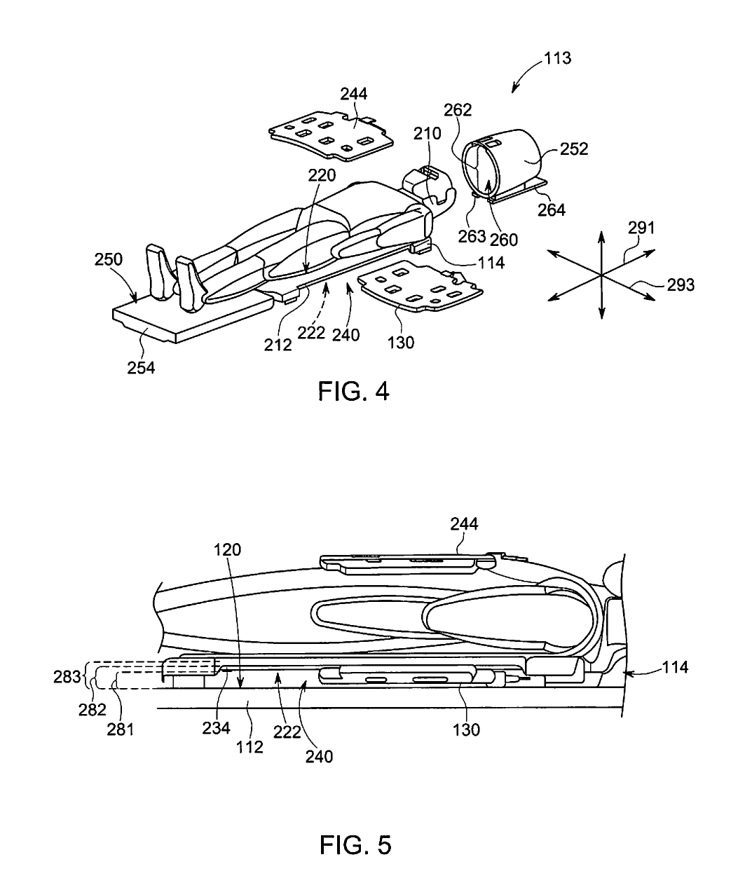

In an exemplary embodiment, the RF coil 130 is a surface coil that is used to image a torso of the patient 102 during an MR imaging session. The RF coil 130 has a plurality of side edges 271-274 that define a substantially panel-like structure. Similar to the top side 220 and the underside 222, the RF coil 130 may have a slightly curved structure such that the RF coil curves about the platform axis 206 when positioned by the underside 222. The RF coil 130 may be communicatively coupled to the MR imaging system. For example, the RF coil 130 may be communicatively coupled to the imaging system 104 (FIG. 1) through a cable 242 (FIG. 3). In particular embodiments, the RF coil 130 is configured to operate in conjunction with another RF coil 244 (shown in FIG. 4) during the imaging session.

However, the RF coil 130 is only exemplary and embodiments described herein may be configured for other types of RF coils. More specifically, embodiments described herein may be used with any RF coil(s) in which it is desirable to remove the RF coil without moving the patient.

Returning to FIG. 2, the head extension 210 extends from the body portion 208 proximate to the leading end 202. The head extension 210 includes a connecting portion 246 and a main (or head) portion 248. The connecting portion 246 extends a distance away from the body portion 208 to the main portion 248. The connecting portion 246 can elevate the main portion 248. In particular embodiments, the connecting portion 246 extends in a substantially perpendicular manner with respect to the top side 220. The head extension 210 also includes a comfort pad 250 that is configured to directly contact the head of the patient 102. As shown in FIG. 2, the head extension 210 has a contoured shape that is configured to receive and hold the head of the patient 102.

In the illustrated embodiment, the platform 114 is formed as a single continuous structure that includes the body portion 208 and the head extension 210. For example, when the composite body material 228 is manufactured and formed (e.g., molded), the composite body material 228 defines both the body portion 208 and the head extension 210. The padding material 229 may then be subsequently added to the body material 228 and the first and second bases 230, 232 may be coupled to the body portion 208. In an alternative embodiment, the first and second bases 230, 232 are formed by the composite body material 228 when the body portion 208 and the head extension 210 are continuously formed. However, the platform 114 may be manufactured in other manners. For example, the body portion 208 and the head extension 210 may be separate parts that are coupled together through an attachment mechanism or frictional engagement.

FIG. 4 is an exploded perspective view of the platform assembly 113 including the platform 114, the RF coils 130, 244 and a leg support 254. In some embodiments, the RF coil 252 may be part of the platform assembly 113. The transfer board 112 (FIG. 1) is not shown. When the platform 114 is mounted onto the transfer board 112, which is supported by the cradle 134 (FIG. 1), the coil-receiving gap 240 is defined between the underside 222 and the support surface 120 (FIG. 1). The RF coil 130 may be moved in a direction along the horizontal axis 293 (i.e., transverse to the longitudinal axis 291) and inserted into the coil-receiving gap 240 from the longitudinal side 212. In an exemplary embodiment, the RF coil 130 may also be inserted into the coil-receiving gap 240 from the longitudinal side 214. The RF coil 130 can be freely supported by the support surface 120. More specifically, a technician may freely insert the RF coil 130 into the coil-receiving gap 240 and place the RF coil 130 onto the support surface 120 where the weight of the RF coil 130 holds the RF coil 130 in place.

In other embodiments, the RF coil 130 may be removably attached to at least one of the support surface 120 or the underside 222. For example, the RF coil 130 may have features that frictionally engage the support surface 120 and/or the underside 222, An attachment mechanism, such as belts, hook and loop fasteners, resilient fingers that grip the RF coil, clamps, and the like, may also be used to removably couple the RF coil 130 to the support surface 120 and/or the underside 222. In some embodiments, it may be possible to removably couple the RF coil 130 to the underside 222 before the platform 114 is removably secured to transfer board 112.

As shown in FIG. 4, the leg support 254 may constitute a separate structure that is independent from the platform 114. For example, the leg support 254 may be a low-profile rectangular block that has a surface 256 that is substantially flush with the top side 220 when the leg support 254 and the platform 114 are mounted onto the transfer board 112. However, in other embodiments, the leg support 254 may be coupled to or formed with the platform 114.

FIG. 4 also illustrates the RF coil 252. In the illustrated embodiment, the RF coil 252 is configured to be used to image the head or neck region of the patient 102. The RF coil 252 may have a substantially circular cross-section with a body-receiving cavity 260 therein that is configured to receive the head of the patient 102. The cavity 260 has an opening 262 that permits the head of the patient 102 to slide therein. The cavity 260 and the opening 262 are sized and shaped to receive the head extension 210 when the head extension 210 is moved in a direction along the longitudinal axis 291. The RF coil 252 has coil supports 263, 264 that are configured to be secured within the imaging bore (not shown) of the gantry. The RF coil 252 is positioned to receive the head of the patient 102 when the patient 102 slides into the imaging bore on the platform 114 with the head of the patient 102 on the head extension 210. In some embodiments, the RF coil 252 is mounted to a patient cradle, such as the patient cradle 134. In such embodiments, the RF coil 252 may receive the head of the patient after the patient slides along the patient cradle on the transfer board.

FIG. 5 is an enlarged side view of the platform 114 on top of the support surface 120 of the transfer board 112. The RF coil 244 is positioned on top of the patient 102 near the ROI. The RF coil 130 has a thickness that permits the RF coil to be inserted into the coil-receiving gap 240. The RF coil 130 is positioned within the coil-receiving gap 240 between the support surface 120 and the underside 222. The RF coil 130 substantially opposes the RF coil 244.

As shown, the contoured section 234 of the underside 222 is separated from the support surface 120 by a separation distance 281. The remainder of the underside 222 is separated from the support surface 120 by a separation distance 282. The side edge 271 of the RF coil 130 extends laterally beyond the longitudinal side 212 of the body portion 208. As shown in FIG. 5, the curved structure of the RF coil 130 may permit the RF coil 130 to clear a bottom surface of the contoured section 234 and clear a bottom surface of the remainder of the underside 222. More specifically, a top of the RF coil 130 may extend a distance away from the support surface 120 that is further than a distance that separates the underside 222 and the support surface 120. As such, the platform 114 may permit portions of the RF coil 130 to be positioned closer to the patient 102.

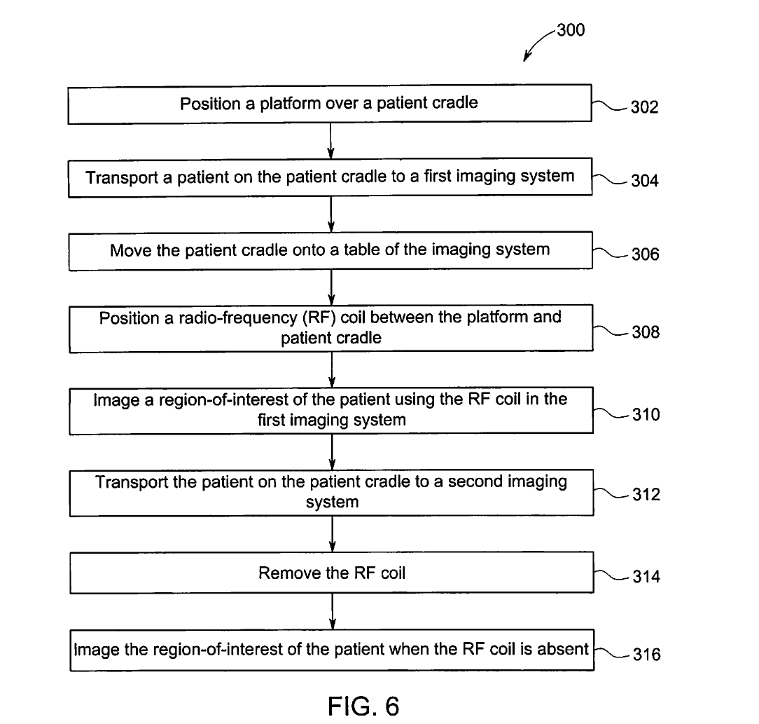

FIG. 6 is a flowchart illustrating a method 300 of imaging a patient. The method 300 may utilize the various apparatuses and systems described above. For example, the method 300 includes positioning at 302 a platform over a body support, such as the transfer board 112, on a medical transporter. The transfer board has a support surface that is configured to receive a patient for imaging. The platform may be similar to the platform 114 and include an underside that faces the support surface. A coil-receiving gap may exist between the underside and the support surface.

The method 300 may also include transporting at 304 a patient on the transfer board to a first imaging system. During transport, the patient can lie on the transfer board with the platform thereon. The first imaging system can be, for example, an MR imaging system or a PET/CT imaging system. The method 300 may also include moving at 306 the transfer board onto a cradle of the first imaging system. The moving 306 may include sliding the transfer board in an axial direction onto the cradle of the imaging system. Alternatively, the transfer board may be lifted off the transport and positioned onto the cradle.

The method 300 may also include positioning at 308 a RF coil within the coil-receiving gap between the underside and the support surface. The RF coil is communicatively coupled to the MR imaging system. The positioning operation at 308 may occur before or after the moving operation at 306. Moreover, the positioning operation at 308 may occur before, after, or while the platform is positioned over the support surface. The RF coil may be positioned within the coil-receiving gap by inserting the RF coil through a side of the transfer board and/or platform into the coil-receiving gap, such as the longitudinal side 212 describe above.

After positioning the RF coil, the method 300 may include imaging at 310 the patient using the MR imaging system. During the imaging, the RF coil may be used to receive signals and transmit signal data to the MR imaging system. After the imaging operation at 310, the method 300 may also include transporting at 312 the patient to a second imaging system that is a different modality than the first imaging system. For example, the second imaging system may be a PET/CT imaging system. The method 300 may also include removing at 314 the RF coil and imaging at 316 the patient with the other imaging system. In some embodiments, it may be appropriate to image the patient with the second imaging system prior to imaging the patient with the first imaging system. In such case, the RF coil is absent from the coil-receiving gap when imaging with the second imaging system.

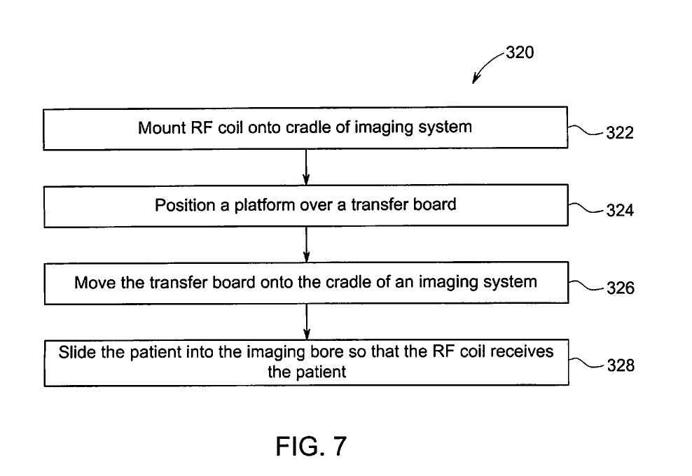

FIG. 7 is a flowchart of another method 320 for imaging a patient. The method 320 may include mounting at 322 a RF coil on a patient table or cradle of an MR imaging system, such as the imaging system 104. The RF coil may be similar to the RF coil 252 shown and described above. The method also includes positioning at 324 a platform over a transfer board, such as the transfer board 112. The transfer board has a support surface that is configured to receive a patient for imaging. The platform includes a head extension that is configured to receive and support a head of the patient.

The method 320 may also include moving at 326 the transfer board onto the patient cradle. The moving 326 may include sliding the transfer board in an axial direction onto the cradle of the imaging system with the patient thereon. The RF coils may be positioned on the cradle to receive the head of the patient. Alternatively, the transfer board may be lifted off the transport and positioned onto the cradle. The method 320 also includes sliding at 328 the transfer board into the imaging bore after the head of the patient and the head extension are received within the RF coil.

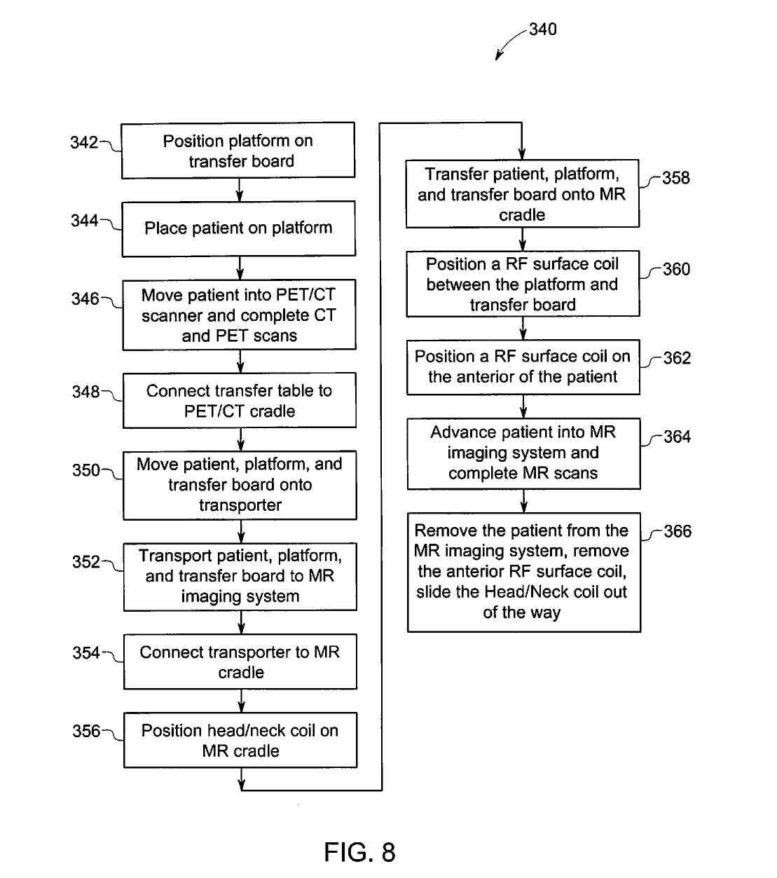

FIG. 8 is a flowchart of a more particular method 340 for imaging a patient. The method 340 includes positioning at 342 a platform onto a transfer board. The transfer board may be positioned onto a patient cradle of a PET/CT scanner. The platform may be removably coupled to the transfer board using, for example, threaded fasteners. The method 340 also includes positioning at 344 a patient onto the platform. The patient may be advanced into a PET/CT scanner at 346 where the patient is scanned to obtain PET images and CT images.

A transporter may be connected to a docking interface of the PET/CT scanner at 348 and, more specifically, to the PET/CT cradle. The transporter is configured to slidably receive the transfer board having the platform and the patient thereon. The transfer board, platform, and patient are moved onto the transporter at 350. The transporter is moved to an MR imaging system at 352 and connected to a docking interface of the MR imaging system at 354.

An RF coil, similar to the RF coil 252, may be positioned onto a patient cradle of the MR imaging system at 356. The patient, platform, and transfer board are then moved onto the patient cradle at 358. The head of the patient may be received by the RF coil that is already positioned on the MR cradle. Another RF coil, such as the RF coil 130, may then be positioned at 360 within a coil-receiving gap between the platform and the transfer board. Another RF coil, such as the RF coil 244, may be positioned onto an anterior of the patient at 362. The patient may then be advanced at 364 into the MR imaging system to obtain MR images. After the MR scanning has occurred, the method 340 may include, at 366, removing the patient from the MR imaging system, removing the anterior RF surface coil, sliding the head/neck RF coil out of the way.

Although the arrows shown in FIGS. 6-8 indicate an order to the different operations in the methods, those skilled in the art understand at least some of the operations may be performed in a different order. For example, with respect to FIG. 6, removing the RF coil at 314 can occur before the transporting of the patient at 312. With respect to FIG. 7, the mounting of the RF coil at 322 can occur after the positioning of the platform at 324.

It is to be understood that the above description is intended to be illustrative, and not restrictive. For example, the above described embodiments (and/or aspects thereof) may be used in combination with each other. In addition, many modifications may be made to adapt a particular situation or material to the teachings of the invention without departing from its scope. For example, the ordering of steps recited in a method need not be performed in a particular order unless explicitly stated or implicitly required (e.g., one step requires the results or a product of a previous step to be available). While the dimensions and types of materials described herein are intended to define the parameters of the invention, they are by no means limiting and are exemplary embodiments. Many other embodiments will be apparent to those of skill in the art upon reviewing and understanding the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A platform configured to be mounted onto a body support, the platform comprising a body portion having an underside and an opposite top side, wherein the underside is configured to face the body support and the top side is configured to support a patient, the body portion also having a coil-receiving recess along the underside, the coil-receiving recess configured to form a coil-receiving gap when the platform is mounted onto the body support, the coil-receiving gap sized and shape to permit a radio-frequency (RF) coil to be positioned therein.

2. The platform of claim 1, wherein the platform has leading and trailing ends and a platform axis that extends therebetween, the body portion including longitudinal sides that extend parallel to the platform axis, the coil-receiving gap being accessible through at least one of the longitudinal sides when the platform is mounted onto the body support.

3. The platform of claim 1, wherein the platform has leading and trailing ends and a platform axis that extends therebetween, the body portion having a substantially uniform cross-section that is taken perpendicular to the platform axis and that extends along the coil-receiving recess.

4. The platform of claim 1, further comprising a head extension that extends from the body portion, the head extension having a contoured shape that is configured to at least partially surround a head of the patient.

5. The platform of claim 4, wherein the head extension has a head side that supports the head of the patient, the head side of the head extension and the top side of the body portion being offset.

6. The platform of claim 1, wherein the platform has a weight and size that permit the platform to be carried and mounted onto the body support by a single individual.

7. The platform of claim 1, further comprising the RF coil, the RF coil having a thickness that permits the RF coil to be inserted into the coil-receiving gap, the RF coil configured to communicatively couple to a magnetic resonance (MR) imaging system.

8. A platform assembly for a body support, comprising:
a platform configured to be mounted onto a support surface of the body support, the platform including an underside that faces the support surface and an opposite top side configured to support a patient, wherein a coil-receiving gap exists between the underside and the support surface when the platform is mounted onto the body support; and
a radio-frequency (RF) coil configured to be positioned between the underside and the support surface in the coil-receiving gap, the RF coil configured to communicatively couple to a magnetic resonance (MR) imaging system.

9. The platform assembly of claim 8, wherein the platform has leading and trailing ends and a platform axis that extends therebetween, the coil-receiving gap being configured to receive the RF coil when the RF coil is moved into the coil-receiving gap in a direction that is transverse to the platform axis.

10. The platform assembly of claim 8, wherein the platform includes first and second bases that project away from the underside, the coil-receiving gap being defined lengthwise between the first and second bases.

11. The platform assembly of claim 10, wherein the platform has leading and trailing ends and a platform axis that extends therebetween, the body portion having a substantially uniform cross-section taken perpendicular to the platform axis along the coil-receiving recess.

12. The platform assembly of claim 8, wherein the platform includes fastener holes configured to receive fasteners for securing the platform to the body support.

13. The platform assembly of claim 8, wherein the platform includes a body portion and a head extension that extends from the body portion, the head extension being located to support a head of the patient and having a contoured shape that at least partially surrounds the head.

14. The platform assembly of claim 8, wherein the platform has a weight and size that permit the platform to be carried by a single individual.

15. The platform assembly of claim 8, further comprising the body support, the body support being a transfer board that is configured to slidably engage a medical transporter.

16. A method of positioning a radio-frequency (RF) coil for imaging a patient, the method comprising:
positioning a platform over a body support having a support surface, the platform including an underside that faces the support surface, wherein a coil-receiving gap exists between the underside and the support surface; and
positioning the RF coil within the coil-receiving gap between the underside and the support surface, the RF coil communicatively coupled to a magnetic resonance (MR) imaging system.

17. The method of claim 16, wherein said positioning the RF coil includes placing the RF coil onto the support surface.

18. The method of claim 16, wherein the RF coil includes a surface coil configured to image a torso of the patient.

19. The method of claim 16, further comprising:
imaging the patient using the MR imaging system; and
imaging the patient using an imaging system of a different modality, wherein the RF coil is absent from the coil-receiving gap when imaging with the imaging system of a different modality.

20. The method of claim 16, further comprising removing the platform from the body support, moving the body support into the MR imaging system, and performing an imaging session.
